(19) United States
(12) Patent Application Publication
(10) Pub. No.: US 2025/0311931 A1
(43) Pub. Date: Oct. 9, 2025

(54) EMERGENCY HEALTH MONITORING SYSTEM AND WEARABLE VITAL SIGN MONITOR

(71) Applicant: Demetrice Williams, Fayetteville, NC (US)

(72) Inventor: Demetrice Williams, Fayetteville, NC (US)

(21) Appl. No.: 19/246,430

(22) Filed: Jun. 23, 2025

Related U.S. Application Data
(63) Continuation-in-part of application No. 17/248,510, filed on Jan. 28, 2021, now abandoned.
(60) Provisional application No. 62/967,407, filed on Jan. 29, 2020.

(51) Int. Cl. A61B 5/021; A61B 5/00; A61B 5/024; A61B 5/11; A61B 5/117; G16H 10/65; G16H 15/00; G16H 20/00

(52) U.S. Cl. CPC A61B 5/021; A61B 5/0022; A61B 5/02405; A61B 5/1112; A61B 5/117; A61B 5/681; A61B 5/742; A61B 5/746; G16H 10/65; G16H 15/00; G16H 20/00; A61B 2503/20; A61B 2562/18

(57) ABSTRACT

An employee health monitoring system includes a quiescent wearable vital sign monitor and a receiving device. The quiescent wearable vital sign monitor has no visual display, no audio output, and no user feedback capabilities, and transmits vital sign data to the receiving device operating as a completely passive monitoring device from the wearer's perspective. Worker health status is monitored by putting the wearable vital sign monitors on workers. The workers' names and work locations are entered into a receiving device having a database. One wearable device is correlated to each of the workers in the database. Vital sign readings are remotely obtained from each of the wearable devices with the receiving device. After determining the health status based on predetermined ranges, workers having normal-range health status are deployed. Workers whose health status is outside of the predetermined normal range are removed from the workplace or rescued.

### CROSS-REFERENCE TO RELATED APPLICATION

This application claims the benefit of priority of U.S. provisional application No. 62/967,407, filed Jan. 29, 2020, the contents of which are herein incorporated by reference.

### BACKGROUND OF THE INVENTION

The present invention relates to an emergency alert system and, more particularly, to a system that monitors individual vital signs to prevent accidents and/or to quickly respond to injury or illness.

Falls from heights require immediate medical attention. In many cases, rescue needs to be completed within 15 minutes to prevent death. Currently, monitoring the safety of workers is done by fellow personnel and/or safety managers. Currently available emergency alert systems have relied on the perception and judgements of humans which can easily be skewed.

Existing wearable health monitoring devices typically incorporate user interface elements such as visual displays, audio alarms, vibration alerts, and other feedback mechanisms designed to inform the wearer of their physiological status, however, these features can be problematic and present significant safety hazards in industrial work environments where worker attention must remain focused on safety-critical tasks, as audio or audible alerts can mask important environmental sounds such as machinery alarms or shouted warnings, visual displays or indicators can divert attention from hazardous conditions requiring constant vigilance, and vibration or haptic feedback can startle workers in precarious positions, potentially causing accidents or falls.

As can be seen, there is a need for an emergency alert system and safety monitoring system that does not rely on subjective human perceptions.

The present invention provides a monitoring device that accurately obtains an unbiased real-time reading of an individual's vital signs and reports the individual's health status to a monitoring system that serves as primary and secondary accident prevention. The inventive device and system allow safety managers and qualified personnel to monitor the heart rate and blood pressure of workers in real time and to be notified if a worker's vital signs fall outside the normal ranges, allowing managers to make informed decisions on whether a worker may continue to work in a specific position or may be removed for safety reasons.

By assessing the health of an individual using vital signs both before and during work, a safety manager may make an informed decision on whether a person is in a suitable condition to work in a specific position. Removal of an employee from the workplace who is not in a suitable state for work may prevent a fall from height or even death.

Moreover, the invention enables emergency services to be contacted as soon as dangerous vital readings are detected. The inventive system saves time when emergency notification is required and provides a real time assessment of the injured worker's condition during rescue. This enables rescue personnel to be better informed when implementing medical procedures and a rescue plan.

The inventive monitoring device and system may be employed in a variety of scenarios. This invention may be used in a school to monitor the health of student athletes, such as football players. The inventive system may be particularly advantageous for athletes subject to extreme heat or cold weather during training and performance exercises, preventing heat exhaustion and other accidents related to health conditions. The inventive monitoring system may also provide real time vital sign monitoring for professional athletes. Soldiers in the military periodically must endure extreme exercises and weather to qualify for duty. The inventive monitoring system may be used to monitor the health vital signs of soldiers training in extreme conditions. The inventive system may also serve as real time vital sign monitoring for medical patients located inside and/or outside of a hospital.

### SUMMARY OF THE INVENTION

In one aspect of the present invention, an employee health monitoring system is provided comprising a quiescent wearable vital sign monitor and a receiving device, wherein the quiescent wearable vital sign monitor has no visual display, no audio output capability, no audible feedback mechanism, and no user interface features, and is operative solely to collect and transmit vital sign data to the receiving device without providing any direct feedback to the wearer.

In another aspect of the present invention, a method of monitoring worker health status is provided, comprising positioning a plurality of quiescent wearable vital sign monitoring devices that have no visual display on a plurality of workers; entering the plurality of workers' names and work locations in a receiving device, having a database, correlating one of the plurality of quiescent wearable vital sign monitoring devices to each of the plurality of workers in the database; remotely obtaining a vital sign reading from each of the plurality of quiescent wearable vital sign monitoring devices with the receiving device; determining whether each vital sign reading indicates a health status within a predetermined normal range or outside of the predetermined normal range; deploying workers having normal-range health status; and removing or rescuing workers whose health status is outside of the predetermined normal range.

The present invention specifically rejects the conventional approach of incorporating user alarm systems, whether audible, visual, or tactile, that are commonly found in health monitoring devices. Unlike systems that include vibrating alarms or other user notification mechanisms, the present invention operates on the principle that worker safety is enhanced by maintaining complete focus on environmental hazards rather than being alerted to physiological conditions that are better managed by remote supervisory personnel.

Surprisingly, eliminating all user feedback mechanisms results in improved worker safety outcomes compared to systems that provide user alerts, as workers maintain better situational awareness of environmental hazards rather than being distracted by device notifications.

These and other features, aspects and advantages of the present invention will become better understood with reference to the following drawings, description, and claims.

### BRIEF DESCRIPTION OF THE DRAWINGS

FIG. 1 is a perspective view of a monitoring device according to an embodiment of the present invention, shown in-use;

FIG. 2 is a perspective view thereof, as well as a receiving device according to an embodiment of the present invention;

FIG. 3 is a front elevation view of the receiving device;

FIG. 4 is a rear elevation view thereof;

FIG. 5 is a sectional view of the monitoring device, taken along line 5-5 on FIG. 2;

FIG. 6 is another perspective view thereof, shown in use;

FIG. 7 is a system diagram according to an embodiment of the present invention; and

FIG. 8 is a flowchart of a method of monitoring employee health according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is an employee health monitoring system comprising a wearable monitor device that monitors a worker's health status by monitoring an employee's vital signs and a receiving device that collects health data sent by the wearable monitor. Preferably, the receiving device collects vital sign data including at least blood pressure data and heart rate data.

The wearable monitor may be worn as a wrist band so the safety manager may record the worker's heart rate and blood pressure (i.e., vital signs). The wristband may comprise monitoring components such as a plurality of diodes operative to read and monitor heart rate and blood pressure of the individual wearing it in real time and a transmission component to transmit the data to the receiving device. The wearable monitor is generally quiescent with no visual display, thereby reducing the risk of distracting the worker.

The device is intended for use in environments where distraction could lead to injury or accidents, such as industrial, construction, or athletic settings.

The wearable monitor is specifically designed without any audio output capabilities, speakers, buzzers, vibration mechanisms, visual indicators, or other user feedback systems commonly found in conventional health monitoring devices. Unlike existing devices that provide audio alerts, visual notifications, or haptic feedback to inform users of their vital signs or health status, the present invention intentionally excludes all such user-facing output features, ensuring the device operates in a completely passive manner from the wearer's perspective. Vital sign data is collected through sensors without alerting, notifying, or otherwise communicating directly with the wearer, and no indication of operational status or collected data is provided. This complete absence of user-perceptible feedback or activity indication is achieved through circuit design that eliminates any components capable of generating perceptible output signals, allowing workers to remain undistracted and fully focused on safety-critical tasks.

The term “passive” in this context means the device does not provide any sensory cues, notifications, or operational status indicators to the wearer under any circumstances.

As used herein, sensors used to collect vital sign data may include, for example, photoplethysmography, pressure sensors, etc.

As used herein, “user feedback systems,” “user-facing output features,” and “user-perceptible feedback or activity indication” refer to any device component or mechanism that provides sensory information to the wearer, including but not limited to audio outputs (e.g., speakers, buzzers), visual outputs (e.g., LEDs, displays), and tactile outputs (e.g., vibration motors, haptic actuators).

This configuration represents a fundamental departure from conventional wearable health monitoring systems, which typically include various combinations of visual displays (light emitting diode [LED] lights, liquid crystal display [LCD] screens, digital readouts), audio outputs (beeps, alarms, voice prompts), and tactile feedback (vibration motors, haptic actuators) to provide real-time health information to the user. The present invention deliberately omits these features to maintain worker focus and prevent distraction in potentially hazardous work environments.

The intentional absence of audio and visual output features in the wearable monitor serves multiple technical and safety purposes. First, it eliminates potential electromagnetic interference that audio circuits and display drivers could introduce into the sensitive vital sign measurement circuits. Second, it reduces power consumption, extending battery life and operational duration. Third, it prevents acoustic masking of critical environmental safety sounds such as equipment alarms, verbal warnings, or approaching machinery. Fourth, it avoids visual distraction that could compromise worker situational awareness in hazardous environments. Fifth, it eliminates the possibility of false alarms or inappropriate responses to device notifications that could endanger worker safety.

The receiving device may be, for example, a portable tablet computer using dedicated software and a Wi-Fi® network and may simultaneously monitor numerous employees wearing the wristbands, thereby providing long-distance, real-time monitoring of worker vital signs. The software may allow continuous updating when improvements are made and addition of applications to improve the performance of the system. The portable tablet may have one or more features in any combination selected from the group consisting of: wireless networking capability, data entry (such as each worker's name and work location), a timer operative to record accident response time, light indicators, one or more universal serial bus (USB) ports, a charging port, an alternating current adapter, a siren alarm, internet access, report generating capability, and downloading capability.

When the information transmitted to a tablet computer and displayed on the screen indicates that the vital signs of a worker are in an appropriate range, the computer may display a green light next to that individual's name. If the software detects a reading that is in a dangerous range, which may occur during a workplace accident, for example, the tablet computer may immediately display a red light next to the worker's name and may initiate an audible alarm. In some cases, the alarm may not stop until it is acknowledged, e.g., reset, by a safety manager or person monitoring the warning system. The safety manager may then implement safety precautions or further safety measures. The computer may start a timer to indicate the amount of time the worker vital signs have been in the dangerous range.

The inventive system and device may be used according to the following method. The construction worker may put a wristband on before starting work. The safety manager may enter the worker's name and work location in the computer tablet database. Before starting work, the safety managers may obtain a reading of the worker's vitals. If the vitals are in an acceptable range, the worker may begin work. If not, the worker may seek medical attention. The wristband may monitor the worker's vital signs throughout the day, transmitting the data to the tablet computer continuously. If a worker becomes ill during work, the worker's vital signs may change sufficiently to set off an alarm, notifying the individual monitoring the system. If a vital reading falls within a danger range, the tablet may notify the safety manager. The safety manager may then decide whether to stop the employee from working or to notify emergency personnel. If an accident occurs, the tablet may start a timer that will inform the safety manager how much time has elapsed since the accident occurred. This information helps the safety manager make informed decisions on an appropriate rescue plan.

The materials of manufacture are not particularly limited. The wristband may be made of a durable, water resistant material. For example, the wristbands may be constructed from neoprene material for durability, water resistance, and ruggedness. The portable tablet computer may be durable and water resistant.

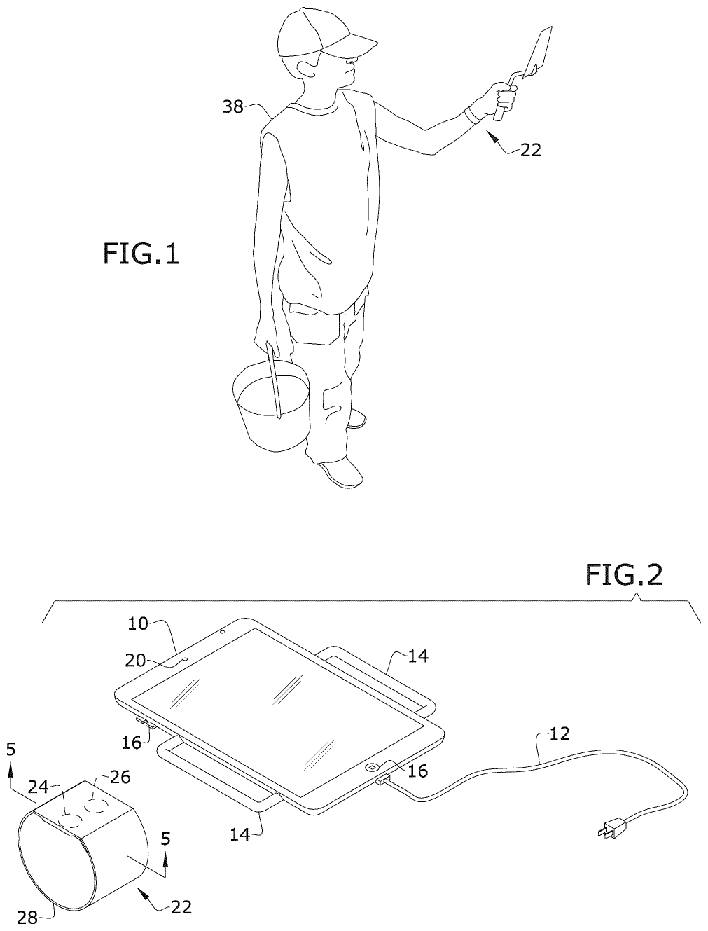

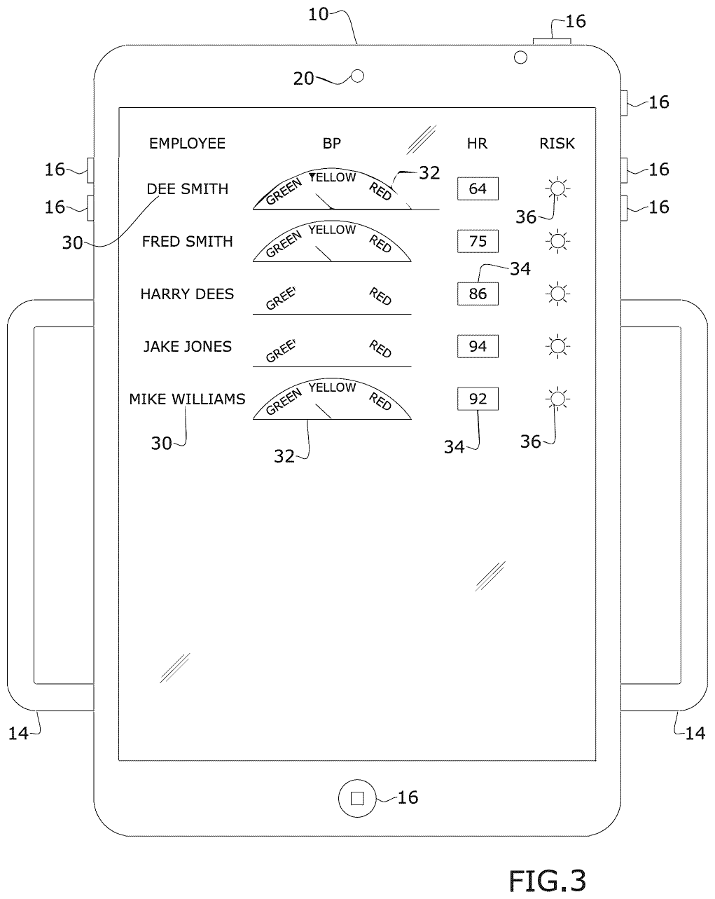

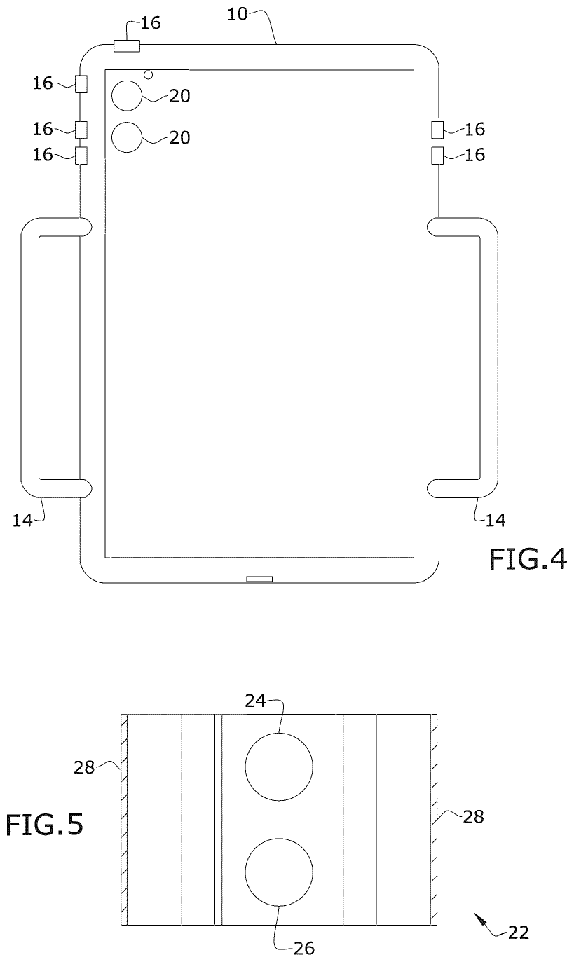

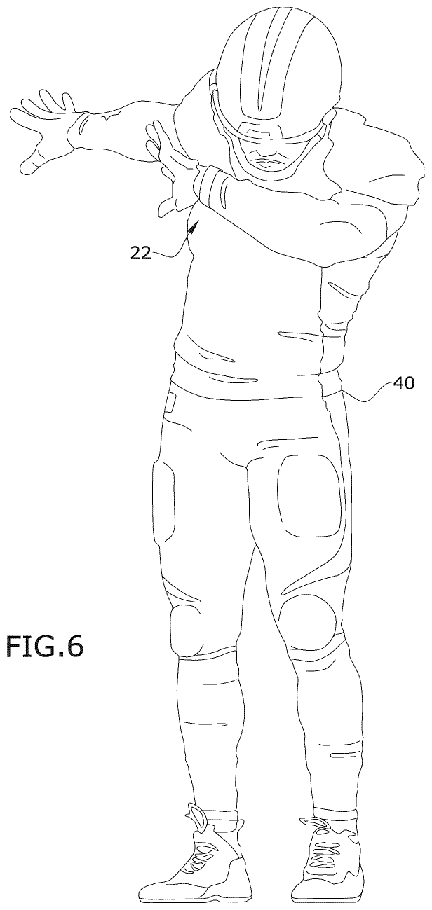

Referring to FIGS. 1 through 8, FIGS. 1 and 6 show a subject, e.g., a construction worker 38 or an athlete 40 wearing a wrist monitor 22 according to an embodiment of the present invention. The inventive wrist monitor 22 is shown in more detail in FIGS. 2 and 5, with a blood pressure sensor 24 and a heart rate sensor 26 attached to a wrist band 28. FIGS. 2 through 4 show a tablet 10 (i.e., a portable Wi-Fi®-capable computer with warning lights, an alarm, and software for displaying worker information). The tablet 10 is shown with a power cord 12, handles 14, buttons 16, a power cord socket 18, and cameras 20. The exemplary tablet 10 is shown with a visual display listing the blood pressure indicators 32 and hear rate readings 34 associated with each employee 30. Risk indicators 36 may illuminate to bring attention to any employee 30 having a vital sign indicating illness, injury, or other distress. For example, an indicator light may show a first color associated with a predetermined normal vital sign range and a second color associated with a predetermined dangerous vital sign range.

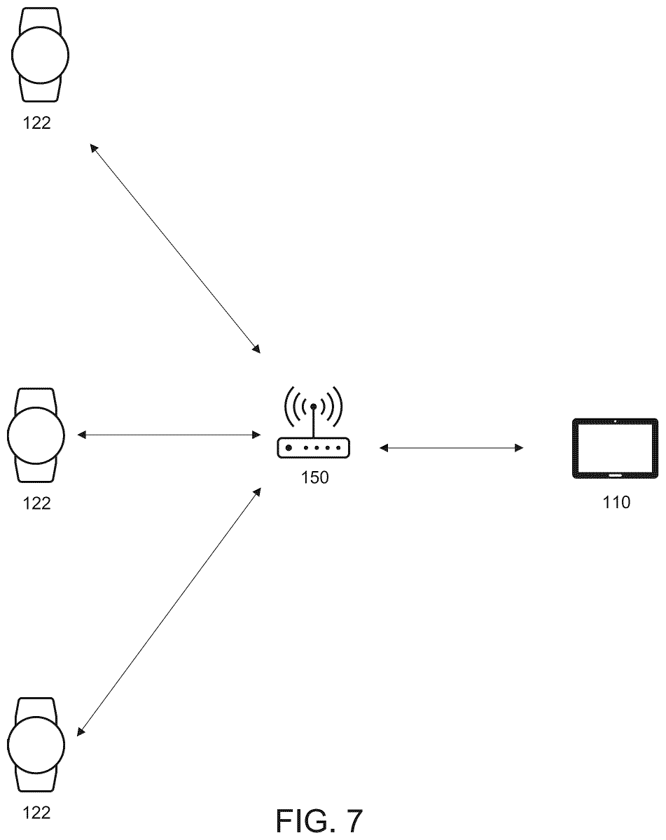

FIG. 7 shows a system according to an embodiment of the present invention, with a plurality of monitored employees 130, each having a wearable monitoring device 122. Each monitoring device communicates with a central monitoring device 110 by way of a wireless network 150.

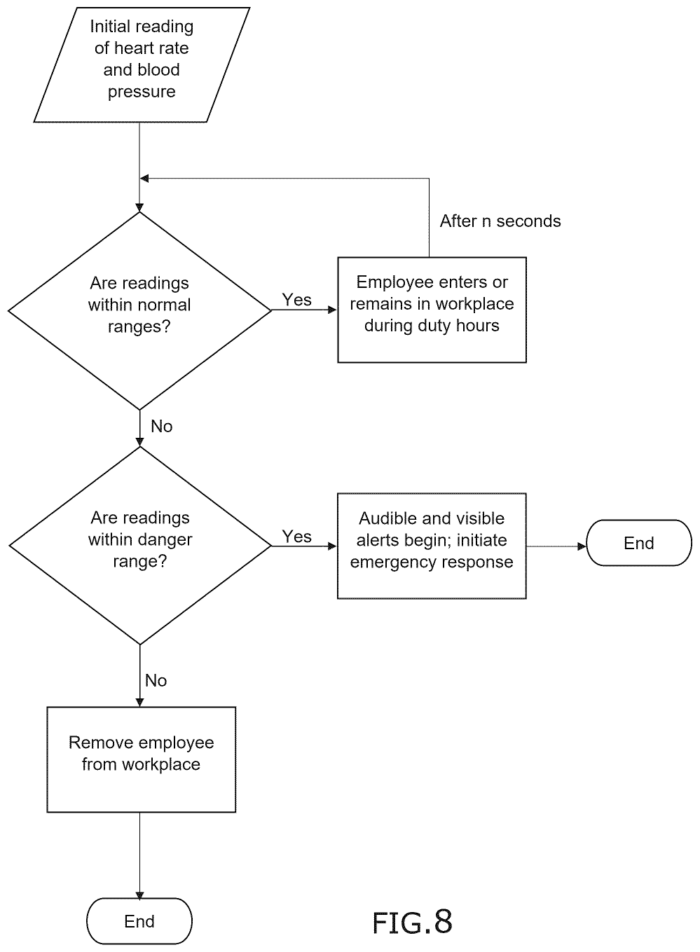

FIG. 8 shows a flowchart process of using the inventive system according to an embodiment of the present invention. After an initial vital sign reading, the results determine whether the employee will enter the workplace or not. Once the worker has entered the workplace, continuous or frequent (e.g., every “n” seconds) vital sign readings indicate the health status of each worker. If the results indicate a health status falls within normal ranges, the employee may remain in the workplace until his or her duty hours end. If the results indicate a health status that falls below or exceeds the normal range, the worker may be removed from the workplace. If the results enter a range predetermined dangerous, audible and visible alerts may begin operative to alert supervising personnel and emergency response may be initiated.

It should be understood, of course, that the foregoing relates to exemplary embodiments of the invention and that modifications may be made without departing from the spirit and scope of the invention as set forth in the following claims.

What is claimed is:

1. An employee health monitoring system, comprising:
a wearable vital sign monitor comprising:
a durable, water-resistant wristband;
a blood pressure monitoring component operative to measure blood pressure;
a wireless transmission component operative to transmit vital sign data; and
a power source; and
a receiving device in wireless communication with the wearable vital sign monitor and comprising a processor and memory;
wherein the wearable vital sign monitor is devoid of audio output capability, visual display capability, haptic feedback capability, and vibration capability, such that the wearable vital sign monitor operates without providing any sensory feedback to a wearer; and
wherein the wireless transmission component is operative to automatically transmit blood pressure data to the receiving device at predetermined intervals.

2. The employee health monitoring system of claim 1, wherein the blood pressure monitoring component further comprises a heart rate monitoring component operative to measure heart rate, and wherein the wireless transmission component is operative to transmit both the blood pressure data and heart rate data to the receiving device.

3. The employee health monitoring system of claim 2, wherein the heart rate monitoring component comprises a plurality of light-emitting diodes and at least one photodetector.

4. The employee health monitoring system of claim 1, wherein the wireless transmission component operates using a protocol selected from the group consisting of: Bluetooth®, Wi-Fi®, Zigbee®, and a proprietary radio frequency protocol.

5. The employee health monitoring system of claim 1, wherein the durable, water-resistant wristband is constructed of neoprene material and includes an adjustable fastening mechanism.

6. The employee health monitoring system of claim 1, wherein the receiving device comprises:
a visual display having at least one green indicator light corresponding to vital sign data within acceptable physiological ranges and at least one red indicator light corresponding to vital sign data outside the acceptable physiological ranges;
an audible alarm operative to generate an alert signal upon receipt of vital sign data outside the acceptable physiological ranges;
a data entry interface; and
at least one data port selected from universal serial bus (USB) ports and charging ports.

7. The employee health monitoring system of claim 6, wherein the receiving device further comprises:
a timer operative to measure elapsed time since the vital sign data was first reported outside the acceptable physiological ranges; and
an internet connectivity component operative to transmit data to remote monitoring systems.

8. The employee health monitoring system of claim 6, wherein the audible alarm, once activated, operates continuously until manually deactivated by authorized personnel through the data entry interface.

9. The employee health monitoring system of claim 1, wherein the receiving device is operative to:
store the vital sign data in a database correlating each wearable vital sign monitor with a specific worker identity;
generate reports based on the stored vital sign data; and
download the stored vital sign data to external devices.

10. The employee health monitoring system of claim 6, wherein the acceptable physiological ranges comprise:
systolic blood pressure between 90-140 mmHg; and/or
diastolic blood pressure between 60-90 mmHg.

11. A method of monitoring worker health status, comprising:
(a) securing a plurality of the wearable vital sign monitor according to claim 1 on a plurality of workers;
(b) entering worker identification data into the receiving device and correlating each wearable vital sign monitor with a respective one of the plurality of workers;
(c) automatically receiving the vital sign data from each wearable vital sign monitor at the receiving device without providing any indication to the plurality of workers;
(d) comparing received vital sign data against predetermined acceptable physiological ranges stored in the receiving device;
(e) activating visual and audible alerts on the receiving device when the vital sign data falls outside the predetermined acceptable physiological ranges;
(f) directing each of the plurality of workers whose vital sign data indicates potential health issues to seek medical evaluation; and
(g) recording a time duration that any of the vital sign data remains outside the acceptable physiological ranges.

12. The method of claim 11, further comprising:
entering work location data for each deployed worker into the receiving device; and
initiating emergency response protocols to specific work locations when the vital sign data from workers at those locations indicates immediate health risks.

13. The method of claim 11, wherein step (c) comprises receiving the vital sign data at intervals ranging from 30 seconds to 5 minutes during active monitoring periods.
